(19) United States
(12) Patent Application Publication
(10) Pub. No.: US 2025/0281544 A1
(43) Pub. Date: Sep. 11, 2025

(54) GENE THERAPY FOR BLEEDING DISORDERS

(71) Applicants: EXPRESSION THERAPEUTICS INC., Tucker, GA (US); CHRISTIAN MEDICAL COLLEGE ASSOCIATION, VELLORE, Vellore (IN); INSTITUTE FOR STEM CELL SCIENCE AND REGENERATIVE MEDICINE, Bangalore (IN); EMORY UNIVERSITY, Atlanta, GA (US); CHILDREN'S HEALTHCARE OF ATLANTA INC, Atlanta, GA (US)

(72) Inventors: Alok Srivastava, Bangalore (IN); Harold Trent Spencer, Tucker, GA (US); Christopher Doering, Tucker, GA (US); Gurbind Singh, Bangalore (IN); Ramachandran Velayudhan Shaji, Bangalore (IN); Gabriela Bout Denning, Tucker, GA (US); John S. Lollar, Atlanta, GA (US)

(21) Appl. No.: 19/073,106

(22) Filed: Mar. 7, 2025

Related U.S. Application Data
(60) Provisional application No. 63/562,988, filed on Mar. 8, 2024.

(51) Int. Cl. A61K 35/28; A61K 31/255; A61K 31/675; A61K 31/7048; A61K 31/7076; A61K 38/37; A61K 39/395; C12N 15/86

(52) U.S. Cl. CPC A61K 35/28; A61K 31/255; A61K 31/675; A61K 31/7048; A61K 31/7076; A61K 38/37; A61K 39/3955; C12N 15/86; C12N 2740/15043

(57) ABSTRACT

Aspects of the present disclosure are directed to a gene therapy approach for treating bleeding disorders, in particular Haemophilia comprising transduction of autologous hematopoietic stem and progenitor cells (HSPCs) with lentiviral viral vectors for expression of blood clotting factors to correct their deficiency in these disorders. The invention further discloses a process and formulation comprising transduced cells for the administration to patients, preferably with bleeding disorders, in particular preceded by a conditioning regimen.

The disclosure also describes myeloablative, non-myeloablative or non-genotoxic conditioning regimens, for preparing the patient for transplantation with formulation of the invention.

### PRIORITY CLAIM

The instant patent application is related to and claims priority from the co-pending US provisional application entitled, “GENE THERAPY FOR BLEEDING DISORDERS”, Application No. 63/562,988, Filed: 8 Mar. 2024, which is incorporated in its entirety herewith to the extent not inconsistent with the description herein.

### TECHNICAL FIELD

The present disclosure relates generally to the field of gene therapy, and more specifically to process, composition, formulation and its use in treatment of bleeding disorders using gene therapy.

The disclosure also describes myeloablative, non-myeloablative or non-genotoxic conditioning regimens, for preparing the patient for transplantation with formulation of the disclosure.

### BACKGROUND

Hereditary blood disorders comprise a wide range of medical conditions such as the major haemoglobin and other red blood cell disorders, clotting factor deficiencies and platelet function disorders, white blood cell or other immune system disorders as well as a range of hematopoietic stem cell related disorders. These disorders arise due to specific genetic mutations which may be amenable to gene therapies. Haemophilia A and B, specifically, are congenital, X-linked bleeding disorders caused by mutations in the genes encoding for proteins called blood clotting factor VIII (FVIII) or factor IX (FIX), respectively. Therefore, people with haemophilia have low levels of these vital proteins that directly correlate with the severity of the disease. Different technologies have been explored to efficiently and safely deliver the therapeutic FVIII and FIX genes into the patient's cells. According to the World Federation of haemophilia, these diseases affect an estimated 400,000 individuals worldwide, of which 80-85% are affected by haemophilia A.

Severe haemophilia can cause spontaneous internal bleeding, and trauma-induced bleeding into muscles and joints, causing chronic pain and reduced function. People with severe haemophilia A have less than 1% of the normal clotting factor levels, leading to life-threatening spontaneous bleeding. A common treatment for haemophilia involves replacing the deficient FVIII and FIX with recombinant or plasma-derived clotting factors. Although these treatments improve the patient's quality of life and significantly prolong life expectancy, the patient remains at risk of developing bleeding episodes and/or chronic joint damage. Additionally, due to the short half-lives of these clotting factors, patients often need frequent treatments, either as prophylaxis or as an immediate response to bleeding episodes, involving the use of repeated doses of FVIII or FIX. Finally, some patients develop neutralizing antibodies in about 20-30% of patients, clinically defined as inhibitors, against the injected proteins that render the treatment ineffective. Adequate clotting factor replacement therapy is accessible to less than 30% of the world population due to its high cost. This treatment also causes major inconvenience due to the need for frequent intravenous injections. Given the limitations of existing protein-based therapies, there is a need to develop a long-term cure for haemophilia. Current research is thus directed towards developing newer, more effective treatments that overcome these challenges, including reducing the frequency of treatments, minimising the risk of inhibitor development, and ensuring wider accessibility and affordability of the treatment.

Recent developments in the field of gene therapy for haemophilia focuses on achieving prolonged high-level expression of clotting factors to stably correct the bleeding disorder. One of the approaches of gene therapy involves using viral or non-viral vectors as vehicles to carry the corrected FVIII or FIX genes into the target cells to achieve long-lasting expression of these proteins. Viral vectors used for gene therapy include adenoviruses, adeno-associated viruses and lentiviruses. A commonly used virus vector for gene therapy for haemophilia is Adeno-associated virus (AAV). Two AAV based products for gene therapy of both haemophilia A and B have received market authorization in 2022. However, in 30-70% of the population, a pre-existing immunity to AAV capsids, potentially excludes a large proportion of individuals from being eligible for this therapy. In this context, alternate technologies are being explored. Lentiviral vectors (LV) holds promise as they are integrating vectors which provide long lasting transgene expression and have been used in haematopoietic stem cell (HSC) directed gene therapies for various immune deficiency disorders as well as the major haemoglobin disorders for nearly two decades. Their use in haemophilia gene therapy is a novel approach also and is being explored.

### SUMMARY

The present disclosure is directed towards development of a drug product candidate for providing biologically active Factor VIII transgene to an individual with defective Factor VIII or limited Factor VIII activity, the method comprising: harvesting autologous mobilized peripheral blood stem cells from the individual; processing the harvested autologous peripheral blood stem cells to obtain an enriched population of hematopoietic stem and progenitor cells; transducing the enriched population of hematopoietic stem and progenitor cells with lentiviral vector such as CD68-ET3-LV or modifications thereof, to obtain a population of transduced cells and; transplanting the transduced cells into the subject with defective Factor VIII gene under conditions appropriate for expression of the Factor VIII protein, wherein said expression results in restoration of Factor VIII biological activity in the individual. The individual with defective Factor VIII may interchangeably referred to as a “patient” or a “donor”.

One aspect of the disclosure relates to a method of providing biologically active factor VIII that involves harvesting the donor's mobilized autologous peripheral blood stem cells, specifically CD34+ cells, which are inherently capable of differentiating into various blood cell types. Mobilizing may involve the use of mobilizing agents that include growth factors such as granulocyte-colony stimulating factor (G-CSF) or granulocyte-macrophage colony-stimulating factor (GM-CSF) or Plerixafor, a chemokine receptor (CXCR4) antagonist. The mobilizing agents stimulate the release of hematopoietic stem and progenitor cells or CD34+ cells from the bone marrow into the circulating blood of the patient. The CD34+ cells are harvested from the mobilised patient blood. The harvested cells are subject to methods known in the art to obtain an enriched population of CD34+ hematopoietic stem and progenitor cells. The enriched population of CD34+ cells are transduced with the Lentiviral vector, CD68-ET3-LV, described in Doering, et al. (2018). The Lentiviral vector disclosed in Doering, et al. carries a Factor VIII transgene designated as ET3 and a CD68 gene promoter that directs monocyte/macrophage-specific expression.

In one embodiment, the method includes pre-stimulating a haemophilia patient's CD34+ cells with as cytokine cocktail, followed by culturing, and transducing the cells in the presence of transduction enhancers capable of increasing vector copy number (VCN). While preclinical studies suggest that VCNs below 0.5 may be sufficient for therapeutic efficiency in animal models, the present disclosure discloses VCNs greater than 1 necessary for effective treatment for gene therapy in humans. In another embodiment, the present disclosure relates to increased FVIII expression in the therapeutic range for the restoration of Factor VIII biological activity in the individual with haemophilia-A, suggesting a positive correlation between VCN and plasma Factor VIII activity.

Another aspect of the disclosure relates to a formulation comprising the haemophilia A patient CD34+ cells transduced with CD68-ET3-LV vector or modifications thereof, hereinafter referred to as CD68-ET3-LV CD34+. The CD68-ET3-LV CD34+ is a genetically modified autologous cell formulation that may be administered back into the individual with a FVIII deficiency, preferably intravenously, at a dosage of at least 2×10⁶ cells/kg of body weight of the individual. In particular, the formulation is for hematopoietic stem-cell engraftment, differentiation into blood cell lineages, and secretion of coagulation factor VIII into the bloodstream, resulting in restoration of the Factor VIII biological activity in the individual with defective factor VIII.

Another aspect of the disclosure relates to a conditioning regimen required for engraftment with CD68-ET3-LV CD34+ to achieve therapeutic levels of factor VIII expression. The conditioning may involve use of myeloablative, non-myeloablative agents or immunosuppressive agents. The agents may be selected from cyclophosphamide, melphalan, Treosulfan, Busulfan, etoposide, Fludarabine, cytarabine, CTLAIg, anti-CD-40L but not limited to these. The agents may further be used in combination for improved conditioning.

### BRIEF DESCRIPTION OF THE DRAWINGS

Various embodiments are illustrated in the figures of the accompanying drawings. Such embodiments are demonstrative and not intended to be exhaustive or exclusive embodiments of the present subject matter.

FIG. 1 is a flowchart diagram illustrating an overview of an exemplary process of gene therapy for bleeding disorders.

FIG. 2 is a table showing the total number of cells and percentage viability from control and transduced samples.

FIG. 3 is a table showing the total number of colony forming units (CFU) from CD34+ cells from control and transduced samples.

FIG. 4 is a table showing the vector copy number (VCN) analysis from transduced samples.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more compositions or elements or structures or components preceded by “comprises . . . a” does not, without more constraints, preclude the existence of other compositions or elements or other structures or other components or additional compositions or additional elements or additional structures or additional components.

Unless otherwise defined, all technical and scientific terms used herein have the same meaning as commonly understood by one of ordinary skill in the art to which this disclosure belongs. The compositions, methods, and examples provided herein are illustrative only and not intended to be limiting.

Embodiments of the present disclosure will be described below in detail with reference to the accompanying drawings.

### I. Definitions

The term hematopoietic stem and progenitor cells (HSPCs) refers to a heterogeneous population of cells within the hematopoietic system that includes both hematopoietic stem cells (HSCs), which have long-term self-renewal capacity, and hematopoietic stem and progenitor cells, which have limited self-renewal potential and primarily give rise to differentiated blood cells. HSPCs reside in the bone marrow and are responsible for generating all myeloid (i.e., monocytes, macrophages, neutrophils, basophils, eosinophils, erythrocytes, dendritic cells, and megakaryocytes or platelets) and lymphoid (i.e., T cells, B cells, and natural killer cells) lineages through the process of haematopoiesis. Unlike fully differentiated blood cells, HSPCs retain the ability to proliferate and differentiate in response to physiological needs or transplantation.

The term peripheral blood mononuclear cells (PBMCs) refer to a group of blood cells, including lymphocytes (T cells, B cells, and NK cells) and monocytes.

The term Human Serum Albumin or HSA refers to a type of serum albumin found in human blood. Albumin is an essential protein which: transports hormones, fatty acids, and other compounds in the blood by acting as a carrier protein; buffers pH; and maintains oncotic pressure.

The term vector refers to a vehicle which is able to carry foreign genetic material into a cell, where it can be replicated and/or expressed. Examples of vectors include non-viral and viral vectors, such as retroviral and lentiviral vectors, which are of particular interest in the present application. Lentiviral vectors, such as those based upon Human Immunodeficiency Virus Type 1 (HIV-1) are widely used as they are able to integrate into the genome of the cells.

The term transgene refers to heterologous or foreign DNA which is not present or not sufficiently expressed in the host cell (i.e. the haematopoietic cell) in which it is introduced. This may include, for example, when a target gene is not expressed correctly in the host cell, therefore a corrected version of the target gene is introduced as the transgene. Therefore, the transgene may be a gene of potential therapeutic interest. The transgene may have been obtained from another cell type, or another species, or prepared synthetically. Alternatively, the transgene may have been obtained from the host cell, but operably linked to regulatory regions which are different to those present in the native gene. Alternatively, the transgene may be a different allele or variant of a gene present in the host cell.

The term transduction as used herein, may be used to describe the insertion of a transgene into the target cell using a viral vector. Transfer of nucleic acid by using viral vector is called transduction. References to transduced cells refer to cells where foreign/heterologous DNA has been introduced into a cell, in particular by a viral vector.

The term transduction enhancer refers to a substance that increases the efficiency of gene transfer into target cells by improving viral vector uptake and integration.

The term autologous as used herein, refers to cells from the same subject. The term allogeneic as used herein, refers to cells of the same species that differ genetically to the cell in comparison.

The term administration refers to administration of a composition to a subject or system to achieve delivery of an agent that is, or is included in, the composition.

Vector copy number (VCN) refers to the average number of lentiviral vector integrations per cell following transduction.

The term CD68 promoter refers to a regulatory DNA sequence that drives gene expression specifically in monocytes and macrophages.

The term engraftment refers to the process by which transplanted hematopoietic stem and progenitor cells (HSPCs) home to the bone marrow, proliferate, and reconstitute the patient's hematopoietic system. Successful engraftment is necessary for long-term Factor VIII production, as transduced CD34+ cells differentiate into monocyte/macrophage lineages that express the therapeutic gene.

The term myeloablative conditioning refers to the administration of high-dose chemotherapy or radiation therapy to eliminate the patient's existing bone marrow cells, allowing the transplanted CD34+ cells to engraft in the bone marrow.

The term non-myeloablative conditioning refers to the administration of a milder dose chemotherapy or radiation therapy that suppresses, but does not fully eliminate, the recipient's bone marrow.

The term immunosuppressive agents refer to drugs that reduce immune system activity to prevent rejection of transplanted cells.

The terms individual, subject and patient are used herein interchangeably. In one embodiment, the subject is a mammal, such as a mouse, a primate, for example a monkey, or a human. In a further embodiment, the subject is a human.

### II. Overview

Aspects of the present disclosure are directed to gene therapy for bleeding disorders. An aspect of the present disclosure is directed to a method for providing biologically active Factor VIII to an individual with defective Factor VIII.

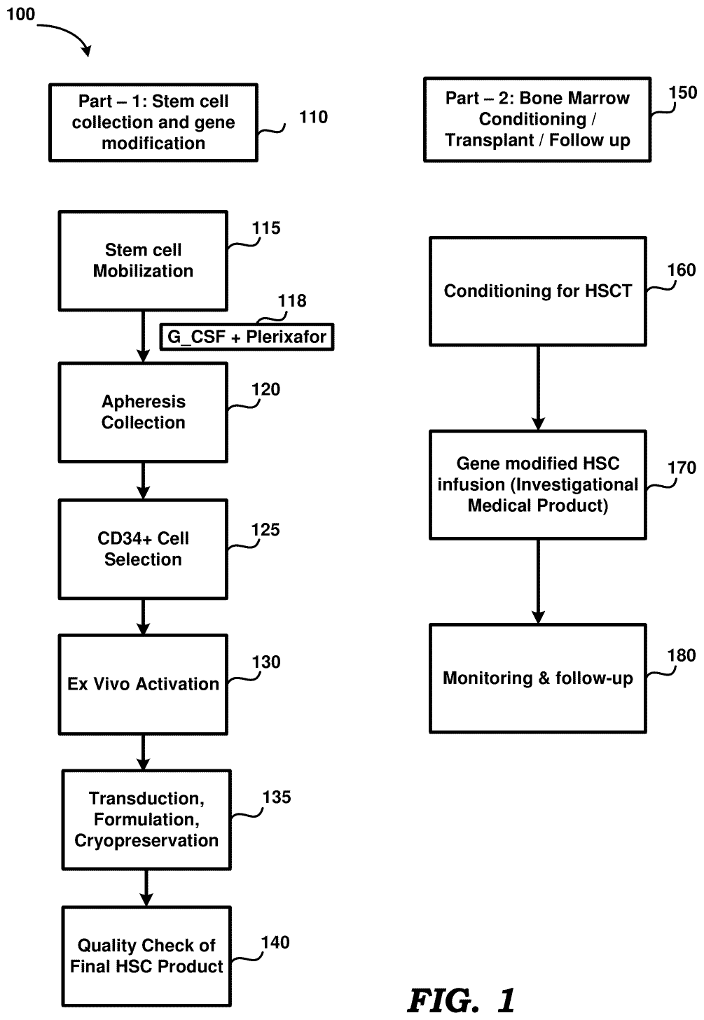

FIG. 1 is a flowchart diagram illustrating gene therapy approach for haemophilia in an embodiment. The overall process (100) consists of Part 1 (110)—manufacturing of transduced cells and Part 2 (150)—bone marrow conditioning, transplantation, and follow-up. The constituent steps of each Part are depicted in the flowchart.

First, autologous peripheral blood stem cells are harvested from the individual with defective Factor VIII. Part 1 (110) begins with the administration of a granulocyte colony-stimulating factor (G-CSF) (118) to the patient, which mobilizes CD34+ hematopoietic stem and progenitor cells (HSPCs) into peripheral blood (PB). Plerixafor may also be administered to enhance mobilization. These mobilizing agents stimulate the release of hematopoietic stem cells to migrate from the bone marrow into peripheral blood, where they can be collected via apheresis.

Following mobilization, the patient undergoes apheresis (120), where peripheral blood mononuclear cells (PBMCs) are collected, and the remaining blood components are returned to the patient. The processing of harvested cells includes separation of mononuclear cells and immunomagnetic selection to obtain an enriched CD34+ population, as detailed in the “Enrichment of autologous CD34+ stem cells” of the Examples section. The collected cells then undergo CD34+ cell selection (125), in which CD34+ cells are enriched through immunomagnetic bead selection to obtain a highly purified population of HSPCs.

These enriched CD34+ cells then undergo ex vivo activation (130), where the CD34+ cells are cultured in specialized media containing growth factors for a particular time period to prepare them for genetic modification. The CD34+ cells then undergo transduction (135) with a monocyte-lineage-restricted, self-inactivating (SIN) lentiviral vector-CD68-ET3-LV, which integrates a functional Factor VIII transgene into the genome of the cells. The vector is designed to drive FVIII expression selectively in monocytes/macrophages, ensuring targeted and sustained production of the clotting factor. The transduction process is enhanced by the inclusion of LentiBOOST, which improves vector copy number (VCN) while maintaining high cell viability. After transduction, the cells are now referred to as CD68-ET3-LV-CD34+ cells. These cells are cultured, formulated and immediately cryopreserved until needed for patient administration.

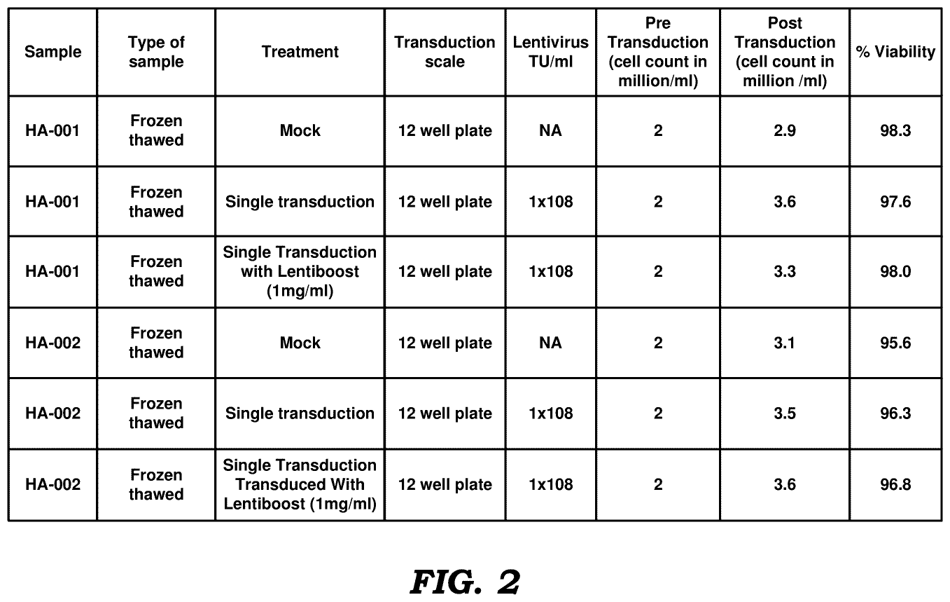

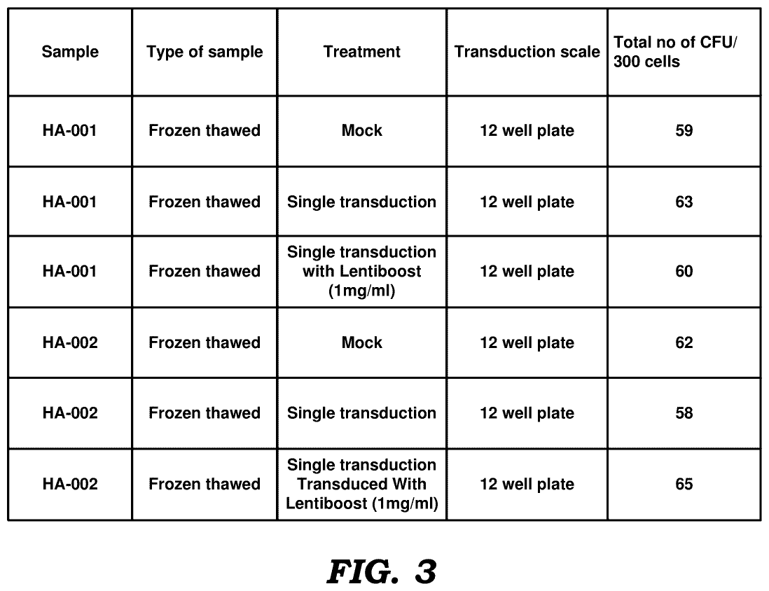

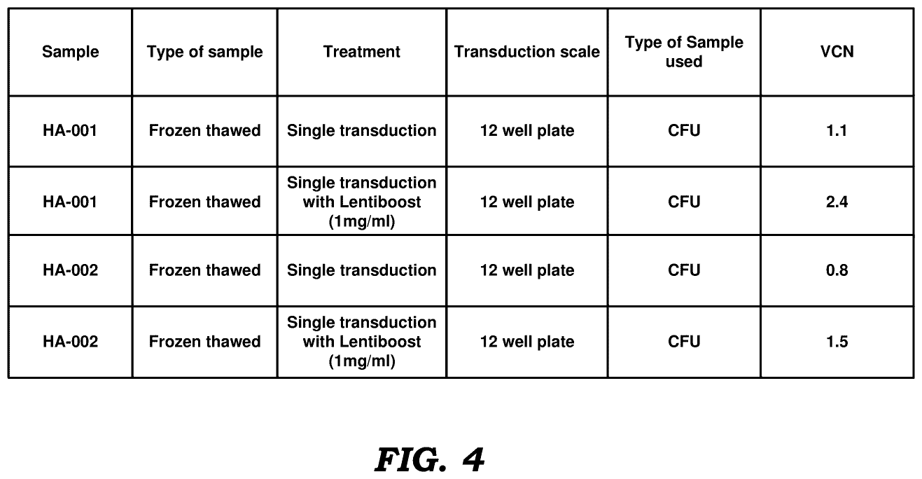

Before administering the cells to the patient, the final CD68-ET3-LV-CD34+ cell product undergoes quality control testing (140), including sterility, cell viability (FIG. 2), colony-forming potential (FIG. 3), and vector copy number analysis (FIG. 4). The quality control testing ensures the transduced product meets the required release criteria before clinical use. If the CD68-ET3-LV-CD34+ cell product meets predefined criteria, the cells are approved for infusion into the patient.

Part 2 (150) involves the conditioning of the bone marrow, clinical administration of CD68-ET3-LV-CD34+ cells, and monitoring of the patient. Before the patient can be infused with CD68-ET3-LV-CD34+ cells, the patient's bone marrow must first be conditioned (160), where the patient receives myeloablative or non-myeloablative agents to prepare the bone marrow environment for engraftment. The conditioning step eliminates existing hematopoietic cells and allows efficient engraftment of the transduced CD34+ cells. Following conditioning, the thawed cryopreserved CD68-ET3-LV-CD34+ cells are infused (170) into the patient intravenously and engraftment occurs. Engraftment is the successful homing, proliferation, and sustained repopulation of the bone marrow by the transplanted CD34+ cells.

The final step involves monitoring and follow-up (180), where patients undergo post-infusion surveillance to assess successful engraftment, Factor VIII expression levels, immune responses, and clinical outcomes over time. Standard monitoring includes periodic FVIII activity assays, vector copy number, and immune profiling to evaluate long-term efficacy and safety. The standard monitoring ensures the therapy's effectiveness and prevention of adverse events.

### III. Transduced Cells

In one embodiment, the transduced cells are hematopoietic stem and progenitor cells (HSPCs). In another embodiment, the HSPCs are CD34+ cells, which are capable of differentiating into various blood cell types. The CD34+ cells are particularly valuable for gene therapy applications in blood disorders such as Haemophilia A due to their ability to self-renew and differentiate into all lineages of the hematopoietic system.

In one embodiment, the CD34+ cells are obtained from peripheral blood (PB) after mobilization. As described in the Examples section, mobilization is achieved through the administration of granulocyte colony-stimulating factor (G-CSF), with or without additional administration Plerixafor. The mobilization agents stimulate the release of CD34+ cells from the bone marrow into peripheral circulation, facilitating their collection through apheresis.

In another embodiment, the CD34+ cells may be obtained from bone marrow directly. The hematopoietic stem and progenitor cells may comprise either bone marrow stem cells, peripheral blood hematopoietic stem and progenitor cells, or a combination of both. The source of CD34+ cells may be from bone marrow, peripheral blood, cord blood and can be selected based on clinical considerations and the specific needs of the individual patient.

In a preferred embodiment, the cells are autologous, meaning they are obtained from the individual with defective who will receive the gene therapy. Autologous cells have the advantage of being immunologically compatible with the recipient, thereby avoiding the need for long-term immunosuppression.

In one embodiment, the CD34+ cells are transduced with the lentiviral vector CD68-ET3-LV, which contains the Factor VIII transgene designated as ET3 and a CD68 gene promoter that directs monocyte/macrophage-specific expression. The CD68-ET3-LV is derived from HIV-1 and has been modified for efficient gene transfer. Lentiviral vectors are particularly advantageous for gene therapy applications due to their ability to integrate into the host genome, providing long-term transgene expression. The CD68-ET3-LV contains nucleic acid sequences encoding a functional Factor VIII protein.

In one embodiment, the transduction process involves the use of transduction enhancers. Transduction enhancers are compounds that improve the efficiency of gene transfer into target cells. In another embodiment, the transduction enhancer is selected from LentiBOOST, PGE2, retronectin, vectofusin, or combinations thereof. In a preferred embodiment, the transduction enhancer is LentiBOOST, which is a non-ionic substance that reduces the microviscosity of the cell membrane, increases lipid exchange, and enhances transmembrane transport. The use of transduction enhancers provides multiple benefits, including increased transduction efficiency, reduced vector requirement, and consequent cost reduction of the manufacturing process, while maintaining cell viability and phenotype.

In one embodiment, the transduction process involves a single transduction method in the presence of enhancers such as LentiBOOST.

### IV. Quality Check of Transduced (CD68-ET3-LV-CD34+) Cells

In an embodiment, cryopreserved CD34+ cells from two Haemophilia A patients who previously underwent apheresis at CMC, Vellore, were used for the transduction process. Cells were transduced with clinical-grade CD68-ET3-lentivirus in the presence of LentiBOOST in a 12-well plate or subjected to mock controls. The transduction conditions, post-transduction cell number, and percentage viability are shown in FIG. 2. FIG. 2 demonstrates that both patient samples (HA-001 and HA-002) maintained cell viability post-transduction, with percent viability ranging from 96.8% to 98.0% for LentiBOOST-treated samples compared to the viability for the mock controls which was 98.3% and 95.6%. Thus, cell viability remained comparable between control and transduced samples in the presence of LentiBOOST. Furthermore, FIG. 2 reveals that post-transduction cell counts increased from the initial 2 million/ml to 3.3-3.6 million/ml across all treatment conditions, indicating cell proliferation despite manipulation.

In another embodiment, a colony-forming unit (CFU) assay was performed on both control and transduced cells, and transduction in the presence of LentiBOOST did not affect the number of CFUs as shown in FIG. 3. Specifically, FIG. 3 shows that CFU counts for HA-001 were 59, 63, and 60 for mock, single transduction, and LentiBOOST-enhanced transduction respectively, while HA-002 showed counts of 62, 58, and 65, demonstrating that LentiBOOST treatment had comparable colony-forming potential. Genomic DNA was isolated from CFU cell pellets obtained from transduced CD34+ cells and from transduced CD34+ cells in the presence of LentiBOOST.

In an embodiment, vector copy number (VCN) analysis was performed using these genomic DNA samples. As shown in FIG. 4, transduced cells in the presence of LentiBOOST exhibited an increased VCN. The VCN enhancement was close to or more than double in both samples (HA-001 and HA-002), with HA-001 showing an increase from 1.1 to 2.4 and HA-002 showing an increase from 0.8 to 1.5 when LentiBOOST was incorporated into the protocol.

These transduction experiments, performed with clinical-grade lentivirus and LentiBOOST, support the single transduction process for future product manufacturing. The viability and colony-forming ability of CD34+ cells remained unaffected by single lentiviral transduction in the presence of LentiBOOST using a clinically relevant protocol. VCN increased in samples from two patients in the presence of LentiBOOST and remained within the desired range for expected therapeutic efficacy. This increase in VCN will reduce the amount of clinical-grade lentivirus required for manufacturing the modified CD34+ cell product. VCN increased in samples from two patients in the presence of LentiBOOST and remained within the desired range for expected therapeutic efficacy. The increased vector copy number correlates with increased Factor VIII expression in differentiated blood cells derived from the transduced CD34+ cells.

### V. Conditioning

In an embodiment, the individual with defective Factor VIII is prepared for the transplantation of CD68-ET3-LV-CD34+ cells through a conditioning regimen using a myeloablative or non-myeloablative agent or an immunosuppressive agent or combination thereof.

In one embodiment, the conditioning regimen involves the use of myeloablative agents. In a further embodiment, the myeloablative agent is selected from the group consisting of Treosulfan, Busulfan, cyclophosphamide, etoposide, and combinations thereof. In a preferred embodiment, the myeloablative agent is Treosulfan.

In another embodiment, the conditioning regimen involves the use of immunosuppressive agents in combination with myeloablative agents. In a further embodiment, the immunosuppressive agent is selected from the group consisting of Fludarabine, CTLA4-Ig, anti-CD40L, anti-thymocyte serum (ATS), anti-thymocyte globulin (ATG), and combinations thereof. In a preferred embodiment, the immunosuppressive agent is Fludarabine.

The conditioning regimen is designed to achieve two primary objectives: (1) ensure efficient engraftment of the transduced CD34+ cells, and (2) induce sufficient immunosuppression to prevent the development of neutralizing antibodies against the Factor VIII transgene product. The combination of myeloablative and immunosuppressive agents has been shown to be effective in achieving both objectives.

In another embodiment, the conditioning regimen may involve the use of non-myeloablative agents. In a further embodiment, the non-myeloablative agents include antibody-directed conditioning targeting specific surface markers such as CD117 or CD45. In yet another embodiment, the conditioning regimen may involve the use of small molecule inhibitors, such as CXCR4 antagonists, as antibody-drug conjugates that reduce off-target effects.

The conditioning regimen will result in expected hematologic events such as neutropenia, thrombocytopenia, and anaemia, as well as non-hematologic events such as mucositis, nausea, vomiting, alopecia, and pyrexia. These events are familiar to transplant physicians and are considered conditioning-related events (CREs). During the conditioning period and until engraftment is established, the individual with defective Factor VIII will maintain therapeutically normal levels of Factor VIII through replacement therapy to prevent bleeding complications.

In a preferred embodiment, the conditioning regimen is administered prior to the infusion of CD68-ET3-LV-CD34+ cells, with the timing of administration optimized to ensure maximal engraftment potential while minimizing toxicity.

In one embodiment, the efficacy of the conditioning regimen may be assessed by monitoring donor cell engraftment, increases in Factor VIII expression levels, and the absence of anti-Factor VIII antibodies. The conditioning protocol may be modified based on the outcomes observed in initial patients to optimize the balance between efficacy and safety.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

### VI. Conclusion

References throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

While various embodiments of the present disclosure have been described above, it should be understood that they have been presented by way of example only, and not limitation. Thus, the breadth and scope of the present disclosure should not be limited by any of the above-described embodiments, but should be defined only in accordance with the following claims and their equivalents.

### EXAMPLES

Mobilisation of Autologous CD34+ Stem Cells from the Patient

The present disclosure defines a method to mobilise hematopoietic and progenitor stem cells in patients with severe bleeding disorders. “Mobilisation” is defined as stimulating the bone marrow using one or more agents like granulocyte colony-stimulating factor (G-CSF) to release stem cells into the bloodstream. In an embodiment, two patients/subjects with severe haemophilia A were first primed with approximately 40-50 IU/kg of extended half-life FVIII concentrate. They were then administered recombinant G-CSF, 5 ug/kg/day, twice a day, subcutaneously for 4 days. The patient then underwent Apheresis with or without administering 0.24 mg/kg Plerixafor. “Apheresis” is defined as a procedure that separates the peripheral blood mononuclear cells from the blood, allowing for its collection while returning the remaining blood components to the donor.

### Enrichment of Autologous CD34+ Stem Cells Using a CliniMACS System

CD34+ cells were enriched from the apheresis product using CliniMACS CD34 magnetic beads. The apheresis product was diluted with CliniMACS buffer containing 0.5% human serum albumin (HSA) and centrifuged at 200 g for 15 minutes. Plasma was removed and the cell pellet was diluted with CliniMACS buffer containing 0.5% HSA. CD34 antibodies were added to the cells and incubated for 30 min at a rocker. These cells were then diluted with CliniMACS buffer containing 0.5% HSA and centrifuged at 300 g for 15 minutes. The resultant cell pellet was resuspended in cliniMACS buffer containing 0.5% HSA and CD34+ cells were collected using CliniMACS plus instrument.

Ex Vivo Transduction of CD34+ Cells with CD68-ET3-LV with an Enhancer

Haematopoietic Stem and Progenitor Cells (HSPC, CD34+) from two patients were pre-stimulated with a cytokine cocktail comprising StemSpan ACF medium containing SCF (stem cell factor), FLT3 (FMS-like tyrosine kinase 3), TPO (thrombopoietin) and IL-3 (Interleukin-3). Cells were centrifuged, and 2 million cells were plated in 12-well plate. Cells underwent single transduction with clinical grade CD68-ET3-LV in the presence of LentiBOOST or subjected to mock transduction (control) in a 12-well plate.

The transduction conditions, post-transduction cell number and percentage viability are shown in FIG. 2. The addition of LentiBOOST did not interfere with the viability of cells as the control and transduced cells had similar viability percentages in the presence of LentiBOOST. FIG. 3 shows the results of colony forming unit (CFU) assay of both control and transduced cells. LentiBOOST did not have any effect on the number of CFUs. Genomic DNA was isolated from CFU cell pellets. Subsequently, VCN analysis was performed from genomic DNA samples. As shown in FIG. 4, VCN was increased in transduced cells in the presence of LentiBOOST. VCN was in the range of 1.5 to 2.4 from genomic DNA of CFU. These transduction experiments performed with clinical grade lentivirus, and LentiBOOST support the single transduction process for future product manufacturing. Transduced cells were cryopreserved using controlled rate cryopreservation and stored in liquid nitrogen.

### Reparation/Conditioning of the Individual for Gene Therapy

In the present disclosure, the patients/subjects are conditioned using a Treosulfan-based myeloablative method to make sure that the subjects maintain therapeutically normal levels of FVIII through replacement therapy during the entire transplant period till engraftment and resolution of any gastrointestinal toxicities. The dosage of Treosulfan was 14 g/m² for 3 consecutive days. While Treosulfan alone may be sufficient for this autologous transplant, a combination with 30 mg/m² Fludarabine for 4 consecutive days to ensure greater immunosuppression and no immune response to the transgene protein is used. Apart from Fludarabine, other myeloablative agents such as Busulfan, cyclophosphamide, and etoposide can also be used in different combinations with Treosulfan and immunosuppressive agents such as CTLA-4-Ig or anti-CD40-L. Non-genotoxic conditioning approaches such as antibody-directed conditioning targeting specific surface markers such as CD117, CD45, and using small molecule inhibitors like CXCL4 antagonists as antibody-drug conjugates that reduce off-target effects can also be used.

The cryopreserved transduced cells were thawed at the bedside of the subject and a single IV injection was performed post-conditioning. A dosage of CD68-ET3-LV-CD34+ of at least 2×10⁶ cells/kg of body weight of the human subject was infused back into subjects.

### US Patent Documents

U.S. Pat. No. 7,635,763 B2 12/2009 Loller et al.

### OTHER PUBLICATIONS

Doering C B, Denning G, Shields J E, Fine E J, Parker E T, Srivastava A, Lollar P, Spencer H T. Preclinical Development of a Hematopoietic Stem and Progenitor Cell Bioengineered Factor VIII Lentiviral Vector Gene Therapy for Hemophilia A. Hum Gene Ther. 2018 October; 29 (10): 1183-1201. doi: 10.1089/hum.2018.137. PMID: 30160169; PMCID: PMC6196756.

What is claimed is:

1. A method for providing biologically active Factor VIII to an individual with defective Factor VIII, the method comprising:
harvesting autologous mobilized peripheral blood stem cells from the individual with defective Factor VIII;
processing the harvested autologous peripheral blood stem cells to obtain an enriched population of hematopoietic stem and progenitor cells;
transducing the enriched population of hematopoietic stem and progenitor cells with Lentiviral vector CD68-ET3-LV or modifications thereof in the presence of a transduction enhancer, to obtain a population of transduced cells; and
transplanting the transduced cells into the subject with defective Factor VIII gene under conditions appropriate for expression of the Factor VIII protein,
wherein said expression results in restoration of the Factor VIII biological activity in the subject.

2. The method of claim 1, wherein the hematopoietic stem and progenitor cells are human CD34+ cells, and wherein the human CD34+ cells undergo pre-stimulation with a cytokine cocktail.

3. The method of claim 1, wherein the transducing the enriched population of hematopoietic stem and progenitor cells is in the presence of an enhancer.

4. The method of claim 3, wherein the transducing the enriched population of hematopoietic stem and progenitor cells in a single transduction method.

5. The method of claim 3, wherein the presence of the enhancer increased the vector copy number or the Factor VIII expression or both in blood cells derived from the hematopoietic stem and progenitor cells.

6. The method of claim 2, wherein the hematopoietic stem and progenitor cells comprises either or both of bone marrow stem cells and peripheral blood hematopoietic stem and progenitor cells.

7. The method of claim 1, wherein the transduced cells are intravenously administered at a dosage of at least 2×10⁶ cells/kg of body weight of the human subject.

8. A pharmaceutical composition comprising the transduced cell population of claim 1.

9. A composition comprising human hematopoietic stem and progenitor cells obtained from subjects with defective Factor VIII and transduced with lentiviral vector CD68-ET3-LV and modifications thereof containing nucleic acid sequences encoding a functional Factor VIII protein.

10. A method for preparing an individual with defective Factor VIII for gene therapy, the method comprising administering at least one or combination of myeloablative and immunosuppressive agent.

11. The method of claim 10, wherein the myeloablative agent is selected from Treosulfan, Busulfan, cyclophosphamide and etoposide.

12. The method of claim 10, wherein the immunosuppressive is fludarabine, CTLA-4-Ig and anti-CD40-L.

13. The method of claim 11, wherein the myeloablative agent is Treosulfan.

14. The method of claim 11, wherein the immunosuppressive agent is Fludarabine.

15. The method of claim 12, wherein the immunosuppressive agent is CTLA-4-Ig.

16. The method of claim 12, wherein the immunosuppressive agent is anti-CD40-L.
